(19) United States
(12) Patent Application Publication
(10) Pub. No.: US 2010/0076520 A1
(43) Pub. Date: Mar. 25, 2010

(54) NEUTRALLY BUOYANT IMPLANTABLE MICROPHONE

(75) Inventor: Scott Allan Miller, III, Lafayette, CO (US)

Correspondence Address: MARSH, FISCHMANN & BREYFOGLE LLP, 8055 East Tufts Avenue, Suite 450, Denver, CO, 80237, US

(73) Assignee: Otologics, LLC, Boulder, CO (US)

(21) Appl. No.: 12/565,622

(22) Filed: Sep. 23, 2009

Related U.S. Application Data
(60) Provisional application No. 61/099,286, filed on Sep. 23, 2008.

(51) Int. Cl. A61N 1/00

(52) U.S. Cl. 607/57

(57) ABSTRACT

An implantable device such as a microphone that may be subcutaneously positioned in surrounding soft tissue. The implantable device may include a hermetically-sealed housing and a diaphragm that forms a portion of an outside surface of the housing. The microphone has a density that is no more than 110% of a density of the surrounding soft tissue. In one arrangement, the device may move in at least substantial unison with the surrounding soft tissue in response to a pressure or compression wave propagating through the soft tissue and being received at the device. In another arrangement, the device may include a filler that may be operable to alter the density of the device.

### RELATED APPLICATIONS

This application claims priority to U.S. Provisional Application Ser. No. 61/099,286 filed Sep. 23, 2008, entitled “NEUTRALLY BUOYANT IMPLANTABLE MICROPHONE,” the entirety of which is hereby incorporated by reference.

### FIELD OF THE INVENTION

The present invention relates to implanted microphone devices, e.g., as employed in hearing aid instruments, and more particularly, to implanted microphone devices.

### BACKGROUND

In the class of hearing aids generally referred to as implantable hearing instruments, some or all of various hearing augmentation componentry is positioned subcutaneously on, within or proximate to a patient's skull, typically at locations proximate the mastoid process. In a fully implantable hearing instrument, typically all of the components, e.g., the microphone, signal processor, and auditory stimulator, are located subcutaneously. In such an arrangement, an implantable auditory stimulator device is utilized to stimulate a component of the patient's auditory system (e.g., tympanic membrane, ossicles and/or cochlea).

By way of example, one type of implantable transducer includes an electromechanical transducer having a magnetic coil that drives a vibratory actuator. The actuator is positioned to interface with and stimulate the ossicular chain of the patient via physical engagement. (See e.g., U.S. Pat. No. 5,702,342). In this regard, one or more bones of the ossicular chain are made to mechanically vibrate causing stimulation of the cochlea through its natural input, the so-called oval window.

As may be appreciated, hearing instruments that utilize an implanted microphone require that the microphone be positioned at a location that facilitates the transcutaneous receipt of ambient acoustic signals. For such purposes, implantable microphones have heretofore been affixed to the skulls of a patient at a location rearward and upward of the patient's ear (e.g., in the mastoid region). Other systems have identified it as being desirable to form a soft tissue mounting where the microphone is removed from the surface of the skull to reduce the receipt and amplification of skull borne vibrations by the implanted microphone.

### SUMMARY OF THE INVENTION

The inventor of the systems and methods (i.e., utilities) provided herein has recognized that, while removal of an implanted microphone from the surface of a patient's bone (e.g., skull surface) may provide some benefits, for example, the attenuation of some forms of biological noise, such soft tissue mounting may raise additional issues. For instance, locating a microphone of an implantable hearing device in soft tissue may subject the microphone to increased sound pressure levels. That is, in some instances, pressure waves may propagate through soft tissue in which the microphone is mounted. Such pressure waves may result from oscillations in the soft tissue caused by, for example, a patient's own voice, chewing, etc.

Irrespective of the cause, pressure or compression waves may propagate through soft tissue, which may result in the soft tissue being displaced. Accordingly, the soft tissue surrounding an implanted microphone may likewise be displaced. In the case of the soft tissue overlying the diaphragm of the microphone, such tissue displacement may be represented as undesired sound in the output signal of the microphone. In this regard, the present inventor has recognized that certain undesired signals in the output of a soft tissue mounted microphone are caused by undesired relative movement between soft tissue and the microphone. The inventor has further recognized that reducing the relative movement between soft tissue and an implanted microphone can reduce the application of undesired sound pressure to the microphone diaphragm and hence reduce the presence of undesired signals in the microphone output.

To reduce the relative movement between an implanted microphone and surrounding soft tissue, the present inventor has recognized that it would be desirable for the implanted microphone to move with the surrounding soft tissue in response to a pressure/compression wave propagating through the tissue. The inventor has also recognized that this co-movement or movement in at least substantial unison may be achieved if the microphone has a density that is substantially equal to and/or no more than about 110% of the density of the surrounding soft tissue. Stated otherwise, it is desirable that the microphone be substantially neutrally buoyant. Neutral buoyancy is a condition where the mass of a physical body equals the mass it displaces in a surrounding medium. In order for the implanted microphone to be near neutral buoyancy or be substantially neutrally buoyant in relation to the surrounding soft tissue, the microphone may have an effective density (e.g., overall density) that is substantially the same as or no more than about 110% of the density of the soft tissue in which it is positioned.

As used herein, a microphone or other implantable device that is “neutrally buoyant” in relation to its surrounding tissue means a microphone or other implantable device with an effective density (e.g., overall density) that is no more than about 110% of the density of the surrounding soft tissue. For instance, as tissue may in some instances have a density that is similar to salt water, or about 1.03 g/cm³, a density of the microphone may be no more than about 1.133 g/cm³. In one variation, the density of the microphone or other implantable device may be no less than about 90% of the density of the surrounding soft tissue. Continuing the above example, the density of the microphone may be no less than about 0.927 g/cm³. In another variation, the density of the microphone or other implantable device may be no more than about 110% of the density of the surrounding soft tissue and no less than about 90% of the density of the surrounding soft tissue.

Generally, the implantable microphone may be formed of a biocompatible hermetically sealed housing such as, for example, titanium or surgical steel. Often a microphone diaphragm may form a portion of the outside surface of the housing. The inside of the housing may include microphone chambers, transducers, electrets, etc. For instance, it may be desirable that the overall mass of the microphone divided by its volume (e.g., its overall density) be similar to the density of patient tissue.

In one arrangement, the implantable microphone may have an overall density of between 0.85 g/cm³ and 1.15 g/cm³ and the surrounding soft tissue may have a density of between 0.95 g/cm³ and 1.05 g/cm³. In another arrangement, the overall density of the microphone may be within a range of between about 0.96 g/cm³ and 1.04 g/cm³.

To achieve a particular density, it may be desirable and/or necessary to fill one or more voids within the housing of the microphone with a filler. For instance, hollow glass beads may be included within the housing to match the overall density of the housing to patient tissue. Other fill materials may be utilized.

### BRIEF DESCRIPTION OF THE DRAWINGS

FIG. 1 illustrates a fully implantable hearing instrument.

FIG. 2 illustrates one embodiment of a soft tissue mount of a microphone.

FIGS. 3A-3C illustrate movement of an implanted microphone in relation to surrounding tissue.

FIGS. 4A-4C illustrate movement of a neutrally buoyant microphone in relation to surrounding tissue.

### DETAILED DESCRIPTION

Reference will now be made to the accompanying drawings, which at least assist in illustrating the various pertinent features of the present invention. The description is not intended to limit the invention to the form disclosed herein. Consequently, variations and modifications commensurate with the following teachings, and skill and knowledge of the relevant art, are within the scope of the present invention. The embodiments described herein are further intended to explain the best modes known of practicing the invention and to enable others skilled in the art to utilize the invention in such, or other embodiments and with various modifications required by the particular application(s) or use(s) of the present invention.

### Exemplary Implantable System

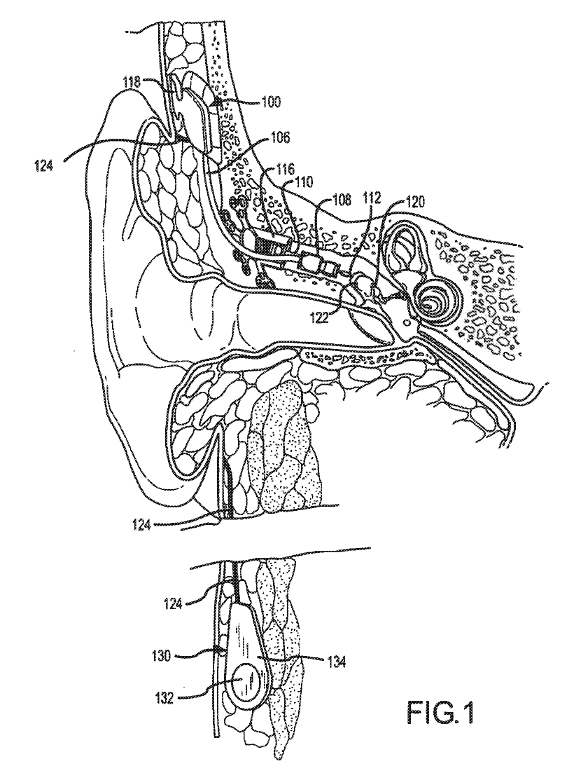

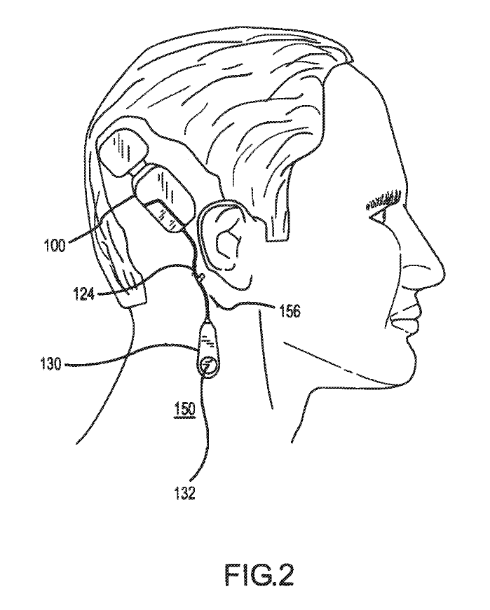

FIGS. 1 and 2 illustrate one application of the present invention. As illustrated, the application comprises a fully implantable hearing instrument system. As will be appreciated, certain aspects of the present invention may be employed in conjunction with semi-implantable hearing instruments as well as fully implantable hearing instruments. Therefore the illustrated application is presented for purposes of illustration and not by way of limitation.

In the illustrated system, a biocompatible implant housing 100 is located subcutaneously on a patient's skull. The implant housing 100 includes a signal receiver 118 (e.g., comprising a coil element) and is interconnected to a microphone assembly 130 via a signal wire 124. The implant housing 100 may be utilized to house a number of components of the implantable hearing instrument. For instance, the implant housing 100 may house an energy storage device and a signal processor. Various additional processing logic and/or circuitry components may also be included in the implant housing 100 as a matter of design choice. In the present arrangement, the signal processor within the implant housing 100 is electrically interconnected via a signal wire 106 to a transducer 108.

The transducer 108 is supportably connected to a positioning system 110, which in turn, is connected to a bone anchor 116 mounted within the patient's mastoid process (e.g., via a hole drilled through the skull). The transducer 108 includes a connection apparatus 112 for connecting the transducer 108 to the ossicles 120 of the patient. In a connected state, the connection apparatus 112 provides a communication path for acoustic stimulation of the ossicles 120, e.g., through transmission of vibrations to the incus 122. To power the fully implantable hearing instrument system of FIG. 1, an external charger (not shown) may be utilized to transcutaneously re-charge an energy storage device within the implant housing 100.

In the present embodiment, the microphone assembly 130 is mounted in soft tissue (e.g., in the neck) such that it is separate and spaced from the implant housing 100 such that it is not mounted to the skull of a patient. The microphone assembly 130 includes a diaphragm 132 that is positioned to receive ambient acoustic signals through overlying tissue, a microphone transducer (not shown) for generating an output signal indicative of the received ambient acoustic signals, and a housing 134 for supporting the diaphragm 132 relative to the transducer. As shown, a wire 124 interconnecting the implant housing 100 and the microphone assembly 130 is routed subcutaneously behind the ear of the patient.

During normal operation, acoustic signals are received subcutaneously at the diaphragm 132 of the microphone assembly 130. The microphone assembly 130 generates an output signal that is indicative of the received acoustic signals. The output signal is provided to the implant housing 100 via a signal wire 124. Upon receipt of the output signal, a signal processor within the implant housing 100 processes the signals to provide a processed audio drive signal via a signal wire 106 to the transducer 108. The audio drive signal causes the transducer 108 to transmit vibrations at acoustic frequencies to the connection apparatus 112 to effect the desired sound sensation via mechanical stimulation of the incus 122 of the patient.

As noted above, the microphone assembly 130 is mounted in soft tissue to isolate the microphone from vibrations carried via the skull of a patient. That is, by spacing the microphone assembly 130 from the skull, vibrations within the skull that may result from, for example, transducer feedback and/or biological sources (e.g., talking and/or chewing) may be attenuated prior to reaching the microphone assembly 130. Stated otherwise, mounting the microphone assembly 130 in soft tissue of the patient may isolate the microphone assembly 130 from one or more sources of non-ambient vibrations (e.g., skull-borne vibrations).

In any soft tissue placement, patient soft tissue is disposed between an underlying bone and the microphone assembly 130. That is, the microphone assembly is not in direct contact with a bone surface as such surfaces may be effective in transferring vibrations to the microphone assembly. In order to maintain the position of the assembly 130 relative to the soft tissue, the assembly may be sutured to such soft tissue. While the soft tissue mount allows for attenuating and/or substantially eliminating the transfer of skull borne vibrations/noise to the microphone assembly 130, it may be desirable to process the microphone output signal(s) to reduce the effect of such noise. One arrangement that may be utilized to reduce the effects of non-ambient sound is described in U.S. patent application Ser. No. 11/330,788 entitled: “Active vibration attenuation for implantable microphone,” having a filing date of Jan. 11, 2006, the entire contents of which are incorporated herein by reference.

While a soft tissue mount of an implanted microphone may provide for attenuation of some forms of biological noise, such microphone mounting may raise additional issues. For instance, it is noted that microphones implanted in soft tissue may be subject to increased sound pressure levels (SPL) due to the movement of surrounding tissue. As will be appreciated, sound propagates as waves of alternating pressure causing local regions of compression and rarefaction. Matter in the medium transmitting the sound is periodically displaced by the wave, and thus oscillates. Generally, the energy carried by the sound wave is split equally between the potential energy of the extra compression of the matter and the kinetic energy of the oscillations of the medium. Stated otherwise, sound is a disturbance of mechanical energy that propagates through matter as a wave (through fluids as a compression wave, and through solids as both compression and shear waves). Particles in the medium are displaced by the wave and oscillate.

As a pressure wave moves through a medium, the medium is compressed and then decompressed as the pressure wave moves away from its origin. In the case of an implanted microphone implanted in soft tissue, the soft tissue is compressed and decompressed. This can result in movement between the soft tissue and the implanted microphone if the microphone does not move in unity with the soft tissue displaced by the pressure wave.

It will be appreciated that relative movement between an implanted microphone and overlying tissue is desirable in the case of acoustic sound. That is, sound impinging on soft tissue overlying an implanted microphone creates a small amplitude displacement through the soft tissue, which is transmitted to the microphone diaphragm underlying that tissue. This causes displacement of the microphone diaphragm, which results in the generation of an output signal that is indicative of the acoustic sound. However, in other cases, pressure waves within the soft tissue may be associated with non-desirable sound sources or vibration sources. For instance, a patient's own voice makes may cause vibrations in soft tissue which results in the generation of larger amplitude pressure waves that pass through the soft tissue. Such non ambient vibrations are often associated with undesirable signals in a microphone output. That is, the relative movement of tissue surrounding the microphone relative to the implanted microphone may result in the application of forces to the diaphragm of the microphone, which are reflected as undesired sound signals within the output signal of the implanted microphone.

FIGS. 3A-3C illustrate the application of forces to an implanted microphone assembly when a compression wave 140 caused by non-ambient vibrations passes through the soft tissue in which the microphone assembly 130 is mounted. As illustrated in FIGS. 3A-3C, each microphone is shown in relation to a reference datum A-A′ for purposes of illustration. As shown in FIG. 3A, the compression wave approaches the microphone assembly 130. As this compression wave passes through the patient tissue, the patient tissue is displaced. As illustrated in FIG. 3B, when the pressure wave 140 passes by the microphone, the microphone may be partially displaced relative to its static position along the reference datum AA. However, the microphone may not displace equally with the surrounding tissue. Accordingly, tissue overlying the diaphragm 132 may be decompressed. In contrast, as the pressure wave/compression wave passes back (e.g., oscillates) through the soft tissue, as illustrated in FIG. 3C, the tissue above the diaphragm 132 may be compressed. As will be appreciated, this decompression and compression of the tissue above the microphone diaphragm results in the application of positive and negative pressures to the microphone diaphragm which are represented as sound signals in the output of the microphone assembly.

The reason that most implantable devices do not move in unison with pressure waves passing through soft tissue is due to the fact that most such devices are much denser than the body tissue and therefore tend to provide an inertial resistance to displacement with the surrounding tissue. Accordingly, it has been determined that by providing an implantable device, such as a microphone assembly, having a density that is substantially similar to and/or no more than a predetermined percentage (e.g., 110%) of the density of the tissue in which it is mounted, the device may be allowed to move more in unison with the surrounding tissue. That is, it may be desirable that the implanted device be at least substantially neutrally buoyant in relation to surrounding tissue.

The density of human tissue may vary by the type of tissue. For instance, fat tissue may have a density of around 0.92 g/cm³ whereas muscle tissue may have a density of approximately 1.04 g/cm³. Accordingly, it may be desirable to determine the density of the tissue at which an implanted device is to be located in order to determine a suitable density for that device. In one arrangement, the density of the device may be no more than about 1.1 g/cm³. In another arrangement, the density of the device may be no less than about 0.9 g/cm³.

In order to substantially alter the density of the microphone assembly 130 in relation to patient tissue, it may be desirable and/or necessary to alter the shape, internal configuration and/or add filler materials to the microphone assembly in order to achieve a desired density. For instance, it may be desirable to add and/or fill voids within the housing of the microphone assembly in order to reduce the overall density of the assembly or implantable device. In one embodiment, voids may be filled or one or more portions of the microphone assembly may be constructed using any appropriate beads or spheres that may or may not be hollow. As an example, these beads or spheres may be 3M™ SCOTCHLITE™ glass bubbles available from 3M Specialty Materials, St. Paul, Minn.

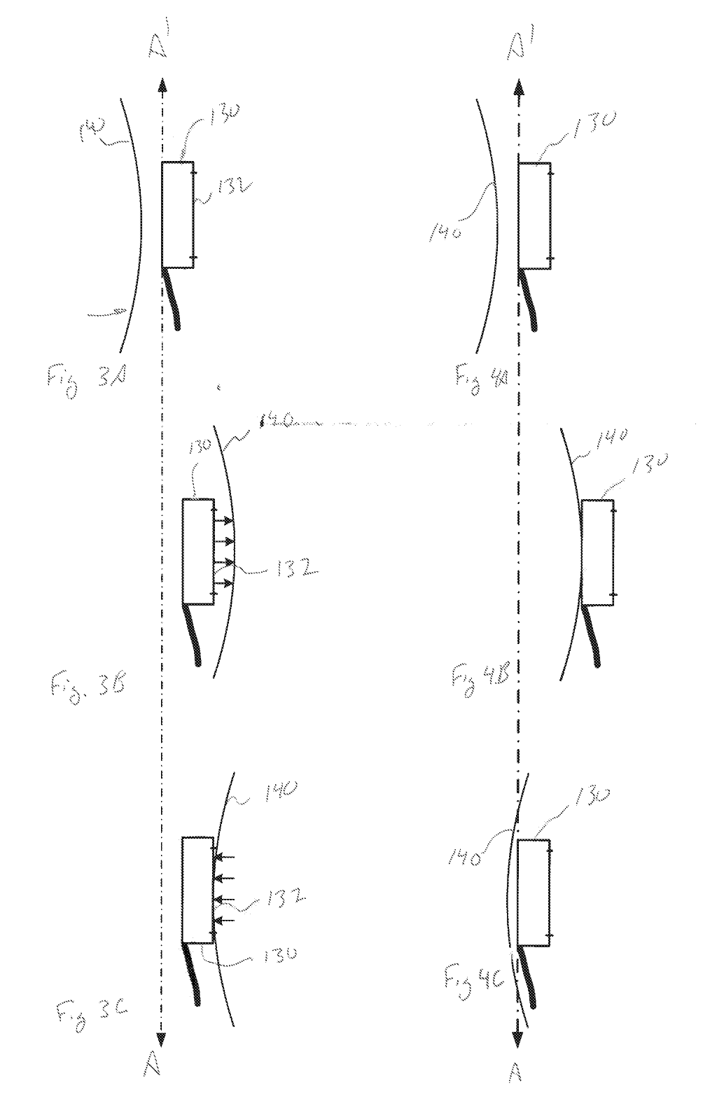

FIGS. 4A-4C illustrate use of a neutrally buoyant implantable microphone. As shown in FIG. 4A, the implantable microphone assembly is positioned along a neutral datum line A-A′ prior to a pressure or compression wave 140 passing through the tissue in which the microphone assembly 130 is positioned. As shown in FIG. 4B and after the compression wave 140 first being received by the microphone assembly 130, the microphone assembly 130 is first displaced in at least substantial unison with the surrounding tissue as the microphone assembly 130 is neutrally buoyant in relation to the surrounding tissue. Thereafter and as the compression wave 140 oscillates back through the tissue and is second received by the microphone assembly 130, the microphone assembly 130 is second displaced in at least substantial unison with the surrounding tissue and may resume its position along the static datum AA′. As will be appreciated, as the microphone assembly 132 has moved in unison with the tissue, there is little or no decompression and/or compression applied to the microphone diaphragm 132 by the compression wave. That is, there is little relative movement between the microphone and the surrounding tissue. Likewise, undesired signals in the output signal of the microphone may be reduced.

The provision of a neutrally buoyant implantable device may provide a further benefit. In this regard, it is noted that when an implantable device is much denser than the surrounding tissue the device has a tendency to migrate (e.g., sink) when implanted. Such migration can increase the local inflammatory response of the body to the device. As previously discussed, a microphone or other implantable device that is “neutrally buoyant” in relation to its surrounding tissue means a microphone or other implantable device with an effective density that is no more than about 110% of the density of the surrounding soft tissue. This may negate the effect of gravity that would otherwise cause the object to sink or migrate subcutaneously. In this regard, an object that has neutral buoyancy may neither sink nor rise. Likewise, when an implantable device has a neutral buoyancy, it is less likely to migrate subcutaneously as the device is supported neutrally with the surrounding medium/tissue. Therefore, use of a neutrally buoyant implantable device may also reduce the anchoring requirements of that device. That is, less force may be applied between the device and surrounding tissue such that fewer sutures or other retention means are required to maintain the device in a desired location.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. For example, in yet another implementation that may realize at least some benefit of the present invention, the microphone may be selected to have a density within a range of about 0.5 g/cm³ to 2.0 g/cm³.

The embodiments described hereinabove are further intended to explain best modes known of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

What is claimed is:

1. An implantable microphone for subcutaneous positioning in surrounding soft tissue, comprising:
a biocompatible, hermetically-sealed housing; and
a diaphragm that forms a portion of an outside surface of the housing, wherein the implantable microphone has a density that is no more than 110% of a density of the surrounding soft tissue.

2. The implantable microphone of claim 1, wherein the implantable microphone is operable to move in unison with the soft tissue in response to pressure waves propagated through the surrounding soft tissue.

3. The implantable microphone of claim 1, wherein the density is no less than 90% of the density of the surrounding soft tissue.

4. The implantable microphone of claim 1, wherein the housing includes a filler.

5. The implantable microphone of claim 4, wherein the filler comprises hollow beads.

6. The implantable microphone of claim 5, wherein the hollow beads comprise glass beads.

7. The implantable microphone of claim 4, wherein the filler is positioned within a void located within the housing.

8. The implantable microphone of claim 1, wherein the implantable microphone has a density of between 0.85 g/cm³ and 1.15 g/cm³ and the surrounding soft tissue has a density of between 0.95 g/cm³ and 1.05 g/cm³.

9. A method for use of an implantable microphone, comprising:
positioning an implantable microphone within surrounding soft tissue at a subcutaneous location;
first receiving a pressure wave at the implantable microphone that has propagated through the soft tissue surrounding the implantable microphone; and
first displacing the implantable microphone in unison with the surrounding soft tissue in response to the first receiving step.

10. The method of claim 9, further comprising after the first displacing step:
second receiving the pressure wave at the implantable microphone that has propagated through the soft tissue surrounding the implantable microphone; and
second displacing the implantable microphone in unison with the surrounding soft tissue in response to the second receiving step.

11. The method of claim 9, further comprising before the positioning step:
adding filler to the implantable microphone so that a density of the implantable microphone is no more than 110% of a density of the surrounding soft tissue.

12. The method of claim 11, wherein the adding step further comprises:
filling one or more voids within a housing of the implantable microphone with the filler.

13. The method of claim 9, further comprising:
suturing the implantable microphone to the surrounding soft tissue.

14. The method of claim 9, wherein a density of the implantable microphone is no more than 110% of a density of the surrounding soft tissue.

15. The method of claim 9, wherein a density of the implantable microphone is no less than 90% of a density of the surrounding soft tissue.

16. The method of claim 9, wherein the implantable microphone has a density of between 0.85 g/cm³ and 1.15 g/cm³ and the surrounding soft tissue has a density of between 0.95 g/cm³ and 1.05 g/cm³.
